(19) United States
(12) Patent Application Publication
(10) Pub. No.: US 2011/0288134 A1
(43) Pub. Date: Nov. 24, 2011

(54) COMPOSITION AND METHOD FOR TREATING FIBROSIS

(75) Inventors: Lola Maksumova, Delta (CA); Douglas Harry Unwin, North Vancouver (CA)

(73) Assignee: Pacific Therapeutics Ltd., Vancouver, BC (CA)

(21) Appl. No.: 13/126,887

(22) PCT Filed: Oct. 28, 2009
(86) PCT No.: PCT/CA2009/001553

§ 371 Date: Aug. 15, 2011

Related U.S. Application Data
(60) Provisional application No. 61/109,446, filed on Oct. 29, 2008.

(51) Int. Cl. A61K 31/198; A61K 31/4418; A61P 15/00; A61P 1/16; A61P 13/12; A61P 9/00; A61K 31/196; A61P 11/00

(52) U.S. Cl. 514/345; 514/563; 514/562

(57) ABSTRACT

The present invention relates, in general, to fibroproliferative disorders, and, in particular, to a method of treating, preventing or reducing fibroproliferative disorders by administering to a mammal in need a composition comprising pharmacologically effective doses of a cytokine modifier, such as tranilast or pirfenidone, and an anti-oxidant which is a precursor of glutathione, such as N-acetyl-cysteine, or their pharmaceutically acceptable derivatives, salts, metabolites, or structural or functional analogues thereof.

### REFERENCE TO RELATED APPLICATION

This application claims the benefit of the filing date of U.S. provisional patent application No. 61/109,446 filed 29 Oct. 2008, the entirety of which is hereby incorporated by reference.

### TECHNICAL FIELD

This invention relates to fibroproliferative diseases. In particular, this invention relates to a composition and method for treatment, prevention and reduction of fibroproliferative diseases.

### BACKGROUND

Fibrosis is the process of forming and developing excessive fibrous connective tissue in an organ or tissue as a reparative or reactive healing response. It is a complex process in which several cellular and biochemical factors modulate the fibrogenesis. Such factors include accumulation of early inflammatory cells, enhanced release of pro-fibrotic cytokines, recruitment of activated fibroblasts, process of trans-differentiation of activated fibroblasts into myofibroblasts; and abnormal regulation of collagen biosynthesis and degradation. Pathological fibrosis, an excessive and abnormal accumulation of collagen, can occur in almost any organ or tissue in the body. Examples include, but are not limited to:

1) all forms of pulmonary fibrosis from coal miners' Black Lung Disease to the treatment-induced varieties occurring in cancer patients and premature babies. Typically fibrocellular scar tissue severely reduces lung diffusion capacity, vital capacity and progresses relentlessly to respiratory failure and death; 2) all forms of liver fibrosis and cirrhosis 3) all forms of vascular fibrosis such as atherosclerosis, peripheral arterial disease and diabetic complications; 4) all forms of renal fibrosis; 5) all forms of interventional therapy triggered fibrosis such as restenosis of blood vessels after balloon angioplasties and atherectomies. These fibroses are the cause of suffering, disability and death in millions of patients across the world. In fact, nearly 45% of all deaths in the developed world are attributed to some type of chronic fibroproliferative disease.

Typically, treatment of fibrosis comprises removal of the underlying cause (e.g., toxin or infectious agent), suppression of inflammation (using, e.g., corticosteroids and immunosuppressive agents such cyclophosphamide and azathioprine), inhibition of fibroblast-like cell proliferation (using colchicines, penicillamine), down-regulation of cytokine machinery (using anti-TGF-β antibodies, endothelin receptor inhibitors, interferons, and others), promotion of matrix degradation (using inhibitors of matrix metalloproteinases), or promotion of fibroblast apoptosis. Despite recent progress, many of these strategies are still in the experimental stage, and existing therapies are largely aimed at suppressing chronic inflammation but lack satisfactory efficacy. Thus, there remains a need for more superior methods and pharmaceutical compositions for treating fibrosis.

Although a great deal of work is still needed to fully understand the mechanisms of fibrosis, a substantial amount of progress has been made in the art to identify major cytokines such as TNF-α and TGF-β1 as the critical players in the fibrotic machinery. Several TNF-α and TGF-β1 modifiers have been developed. Among those are tryptophan derivative such as tranilast and pyridone derivative such as pirfenidone.

Tranilast (n-[3,4-dimethoxycinnamoyl] anthranilic acid) is an orally administered anti-allergic drug used widely in Japan and Korea for bronchial asthma, allergic rhinitis and atopic dermatitis. However, it also has potent anti-fibrotic effects demonstrated in various animal models of fibro-proliferative disorders. The mechanisms of tranilast's antifibrotic effects are not fully understood, but a major mode of action appears to be the suppression of the expression and action of TGFβ-1. Notably, many years of clinical use have revealed that tranilast is safe and well tolerated at doses of up to 600 mg/day for at least 3 months representing a major advantage over other drugs currently in the early or mid-phase of drug development in fibrosis indication.

Pirfenidone (5-methyl-L-phenyl-2-(1H)-pyridone), a small molecule compound initially developed as anthelmintic drug, has been reported to have beneficial effects for the treatment of certain fibrotic diseases. The efficacy of anti-fibrotic activity of pirfenidone has been further demonstrated in various animal models and human trials.

On the other hand, it is universally accepted that oxidative stress (imbalance between oxidants and antioxidants) plays a key role in the pathogenesis of miscellaneous diseases including pathological fibrosis. Antioxidant supplementation has been studied extensively as a method to counter disease-associated oxidative stress. Several antioxidants have been used with varying degrees of success. Commonly used antioxidants include vitamin C, vitamin E, vitamin K and lipoic acid. However, the cysteine prodrug N-acetyl-cysteine (NAC), has proven to be effective in treating fibrosis diseases (Demedts, Behr et al. 2005).

The above mentioned drugs (tranilast, pirfenidone and NAC) in order to show an effect in fibroproliferative disorders need to be administered in high customary doses. These dosages elicit undesired and serious side effects. The present invention overcomes limitations in the prior art and addresses a need for pharmaceutical compositions that combine these active components that act synergistically to achieve strong anti-fibrotic effect with greater improvement in the general tolerability.

### SUMMARY OF THE INVENTION

Methods and compositions for treating, preventing, or reducing fibroproliferative disorders, as well as delaying disease progression associated therewith are provided. In one embodiment, the method includes administering a composition comprising an anti-oxidant which is a precursor of glutathione and a second agent selected from the list of TNF-alpha and/or TGF-β1 modifiers. The modifiers may be tranilast or pirfenidone, or their pharmaceutically acceptable salts, derivatives, metabolites or structural or functional analogues thereof. These agents are present in the amounts that, when administered to a mammal in need, are sufficient to reduce fibrosis process. The composition may be formulated for topical or systemic administration. In one embodiment of the invention, the anti-oxidant is N-acetyl-cysteine and the second agent is tranilast or pirfenidone. In another embodiment of the invention, the composition comprises pharmaceutically acceptable salts, derivatives, or structural or functional metabolites of either or both of the anti-oxidant and the cytokine modifier.

A composition comprising a pharmacologically effective dose of an anti-oxidant which is a precursor of glutathione and a pharmacologically effective dose of the cytokine modifier is also provided according to the present invention. In some embodiments, the cytokine modifier is tranilast or pirfenidone and the anti-oxidant is N-acetyl-L-cysteine. In other embodiments, the composition comprises pharmaceutically acceptable salts, structural or functional analogues, derivatives or metabolites of either or both of the cytokine modifier.

### BRIEF DESCRIPTION OF DRAWINGS

In drawings which show non-limiting embodiments of the invention:

FIG. 1 illustrates the effect of tranilast (A), pirfenidone (B) and N-acetyl-cysteine (C) on TGF-β1 induced extracellular matrix accumulation. Left panel represents Sirius Red optical density readings from 6 replicate wells. Right panel depicts relative % inhibition of TGF-β1 induced ECM accumulation by each compound.

FIG. 2 illustrates % inhibition of TGF-β1 mediated ECM accumulation by the pharmaceutical composition of a combination of tranilast with N-acetyl-cysteine. A range of therapeutic concentrations of tranilast (1-300 μM) was mixed with a range of NAC concentrations (0.1-20 mM) in the screen plate as shown in A. Synergistic efficacy of the most promising combination was confirmed in the second run with n=6 as shown in B.

FIG. 3 illustrates % inhibition of TGF-β1 mediated ECM accumulation by the pharmaceutical composition of a combination of pirfenidone with N-acetyl-cysteine. A range of therapeutic concentrations of pirfenidone (10-1000 μM) was mixed with a range of NAC concentrations (0.1-20 mM) in the screen plate as shown in A. Synergistic efficacy of the most promising combination was confirmed in the second run with n=6 as shown in B.

### DESCRIPTION

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and drawings are to be regarded in an illustrative, rather than a restrictive, sense.

The inventors have shown that cytokine modifiers such as tranilast or pirfenidone in combination with an anti-oxidant/precursor of glutathione such as N-acetyl-cysteine exhibit substantial synergistic and super-additive anti-fibrotic effect in TGF-β1 mediated collagen synthesis by human lung fibroblasts.

TGF-β1 is a major mediator of fibroproliferative disease. Therefore, suppression of pro-fibrotic cytokines using a combination of tranilast and NAC or a combination of pirfenidone and NAC can be successfully used to treat fibroproliferative disorders. The inventors have found that tranilast plus NAC or pirfenidone plus NAC combinations of the invention result in the enhancement of the anti-fibrotic activity of the tranilast and pirfenidone by several folds when the said compound is combined with a subtherapeutic dose of NAC, even when NAC is administered at a dose lower than that at which it is known to be effective as an anti-oxidant. For example, pirfenidone is often administered at 1800 mg/day orally, while NAC is generally taken in amounts between 1200-1800 mg/day. The inventors have shown a several fold increase in the potency and safety of the pirfenidone by combining it, at 600 mg/day, with 600 mg/day NAC.

Accordingly, in one embodiment the present invention relates to a method of treating fibroproliferative disorders in mammals. In one embodiment, the method comprises administering to a mammal in need of such treatment an effective amount of a composition comprising an effective dose of tranilast or pirfenidone and N -acetyl-L-cysteine. Structural and functional analogs of each of these compounds are known, and any of these analogs can be used in the anti-fibrotic combination.

The terms “treat” and “treatment” are used broadly to denote therapeutic and prophylactic interventions that favourably alter a pathological state. Treatments include procedures that moderate or reverse the progression of, reduce the severity of, prevent, or cure a disease. As used herein, the term “fibroproliferative” includes all forms of pulmonary (idiopathic, occupational and environmental, auto-immune, scleroderma, sarcoidosis, drug- and radiation-induced, genetic/familal fibrosis); all forms of liver fibrosis and cirrhosis; all forms of kidney fibrosis, all forms of uterine fibrosis; all forms of vascular fibrosis such as atherosclerosis and diabetic complications; all forms of interventional therapy triggered fibrosis such as restenosis of blood vessels after balloon angioplasties and atherectomies.

Preferred active agents include either tranilast or pirfenidone or any pharmaceutically acceptable derivatives or metabolites thereof, as well as any structural or functional analogs thereof. While the use of N-acetyl-L-cysteine is also preferred, other precursor compounds that replenish glutathione concentration in the tissue or body cavity can be used, for example NAC amide, cysteine esters, gammaglutamylcysteine and its ethyl ester, glutathione derivatives such as glutathione monoester, glutathione diester, lipoic acid and derivatives thereof can be used. Pharmaceutically acceptable derivatives, metabolites or structural and functional analogs of N-acetyl-L-cysteine may also be used.

The amount of active agents (e.g., tranilast, pirfenidone and N-acetyl-L-cysteine) administered can vary with the patient, the route of administration and the result sought. Optimum dosing regimens for particular patients can be readily determined by one skilled in the art. For example, the daily dose of tranilast can be from 100 mg to 600 mg combined with a daily dose of N-acetyl-L-cysteine from 200 mg to 1800 mg. The daily dose of pirfenidone can be from 100 mg to 1200 mg. The ratio of tranilast or pirfenidone to N-acetyl-L-cysteine can also range. Administration of each compound of the combination may be by any dose ratio that results in a concentration of the compound that, combined with the other compound, is anti-fibrotic (i.e. a pharmacologically effective dose).

The individual components of the composition can be administered separately at different times during the course of therapy or concurrently in divided or single combination forms.

The active agents (which may be, for example, tranilast or pirfenidone in combination with N-acetyl-L-cysteine) can be administered in any convenient manner, such as orally, by inhalation, sub lingually, rectally, parenterally (including subcutaneously, intrathecally, intramuscularly or intravenously), or transdermally.

The active agents may be administered in the form of a pharmaceutical composition or compositions that contain one or both in an admixture with a pharmaceutical carrier. Each compound is admixed with a suitable carrier substance, and is generally present in an amount of 1-95% by weight of the total weight of the composition. The pharmaceutical composition can be in dosage unit form such as tablet, capsule, sprinkle capsule, pill, granule, powder, syrup, suspension, emulsion, solution, gel, paste, ointment, cream, lotion, plaster, drench, suppository, enema, injectable, implant, spray or aerosol. The composition can also be present in a transdermal delivery system, which may be, by way of example, a skin patch.

A large variety of delivery vehicles for administering the composition are contemplated as within the scope of the present invention when containing therapeutic amounts of cytokine modifier (for example, tranilast or pirfenidone) and antioxidant (for example, NAC). Suitable delivery vehicles include, but are not limited to, microcapsules or microspheres; liposomes and other lipid-based release systems; absorbable and/or biodegradable mechanical barriers, polymeric or gel-like materials.

The pharmaceutical compositions may be formulated according to conventional pharmaceutical practice. Sustained release formulations can also be used. Each compound of the combination may be formulated in a variety of ways that are known in the art. For example, the first agent (cytokine modifier) and the second agent (anti-oxidant) may be formulated together or separately. Desirably, the two components are formulated together for simultaneous administration. Such co-formulated compositions can include the two agents formulated together in the same pills, capsule, liquid, etc. The individually or separately formulated agents can be packaged together as a co-packaged product. Non-limiting examples include two pills, a pill and a powder, a suppository and a liquid in a vial, two topical creams, etc.

A composition of a cytokine modifier (such as tranilast or pirfenidone) and an anti-oxidant that replenishes glutathione in tissues (such as N-acetyl-L-cysteine) is an effective treatment for fibroproliferative disorders and provides an effective means of delaying disease progression associated with fibrosis. The composition can be more effective than, for example, tranilast or N-acetyl-L-cysteine treatment alone and with fewer side effects. Lower doses of both types of medication can be used in the compound treatment, thereby further reducing the overall side effect burden. It is a particular advantage that, because of synergistic and super-additive effect on administration, the amounts of tranilast (or pirfenidone) and N-acetyl-L-cysteine which are to be administered can be reduced to those amounts which, on administration alone, show only a minimal pharmacological effects so that, at the same time, side effects which are elicited by high doses of these medicaments can be diminished. This is of great importance because it is known that N-acetyl- L-cysteine can, in the customary doses, elicit undesired side effects such as nausea, vomiting, headache, dry mouth, dizziness, or abdominal pain (Whyte, Francis et al. 2007). Tranilast may show undesired side effects in the liver (elevation of transaminase level with almost two times the healthy limit and jaundice), digestive system (abdominal discomfort, nausea, vomiting, diarrhea, and so on), skin (rash and itching), and urinary system (frequent urination and cystitis) (Holmes, Fitzgerald et al. 2000; Azuma, Nukiwa et al. 2005). The most common side effects of pirfenidone include a rash and sun sensitivity, nausea, vomiting, loss of appetite, drowsiness, and fatigue (Azuma, Nukiwa et al. 2005). When used in combination, it is now possible to reduce drastically the dose of tranilast or pirfenidone necessary for humans, as well as the amount of N-acetyl-cysteine below the dose of each compound that would be pharmacologically effective when the compound is used in isolation, so that there is an even greater improvement in the general toxicological tolerability with therapeutic efficacy.

As will be apparent to those skilled in the art in the light of the foregoing disclosure, many alterations and modifications are possible in the practice of this invention without departing from the spirit or scope thereof.

### EXAMPLE 1

### In Vitro Investigations

The composition comprising tranilast and NAC or composition containing pirfenidone and NAC was investigated for their antifibrotic activity by employing in vitro collagen synthesis assay: the TGF-β1 induced monolayer extracellular matrix (ECM) accumulation assay in fibronectin-coated plates.

Method Human lung fibroblast cell line, HFL1, was purchased from American Type Culture Collection. Cells were maintained in FK12 medium supplemented with 10% FBS and antibiotics. Cells were trypsinized and seeded into 96-well fibronectin-coated plate as 5×10⁴cells/well and cultured overnight to achieve 60-80% confluence. After a washing with PBS and serum-free medium, fresh medium supplemented with 40 pM of TGF-β1 was added in each test well. Different concentrations of tranilast, pirfenidone, NAC and their combinations were also added to some test wells. The plates were left at 37° C. in a CO₂ incubator for 72 h. After removing medium cells were fixed to the plate with 0.5% glutaraldehyde for 30 minutes at room temperature. Cells were washed and treated with 0.5M acetic acid for 30 minutes. After washing cells with distilled water 250u1 of Sirius Red was added for 2 hours. Dye was removed and cells were washed with distilled water. Sirius Red was eluted with 200 ul 0.1 N sodium hydroxide, and the optical density at 540 nm was determined using a Molecular Dynamics spectrophotometer.

Results

Effect of Tranilast, Pirfenidone and NAC on TGF-β1 mediated ECM accumulation

The inventors first examined the effect of the three therapeutic compounds: tranilast, pirfenidone and NAC in the above described experimental methodology. In the negative control (in the absence of exogenous TGF-β1 picro-Sirius red-positive collagen was limited. In contrast, addition of exogenous human TGF-β1 induced deposition of picro-Sirius red-positive collagen by 2 fold (0.189±0.012 vs. 0.094±0.005; P<0.0001).

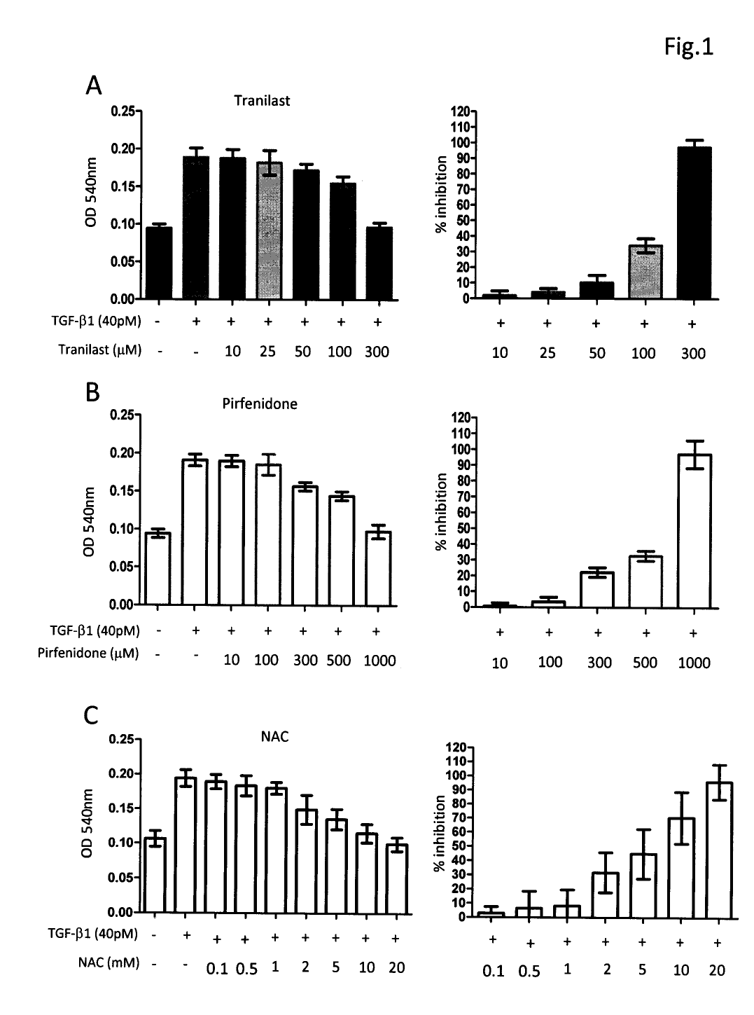

Tranilast is an anti-allergic drug widely used in Japan and Korea for keloids and scleroderma. After oral administration of the usual therapeutic dose of 600 mg/day tranilast, the plasma concentration has been reported to reach 30-300 μM (Kusama, Kikuchi et al. 1999). The inventors tested a range of concentrations of tranilast (10-300 uM) on collagen accumulation in HFL1 cells stimulated by exogenous human TGF-β1. Tranilast has effectively abrogated TGF-β1 mediated ECM in a dose-dependent manner, whereas tranilast at lower concentrations, i.e. 10, 25, 50 μM, showed no significant inhibition; 100 μM tranilast demonstrated 34±11% (P<0.005) inhibition; and at 300 μM inhibition has reached 100% (P<0.0001) (FIG. 1A).

Pirfenidone has been reportedly tested with promising results in patients with idiopathic pulmonary fibrosis. The usual therapeutic dose of 1200 mg/day yields the plasma concentration of 100-1000 μM (Shi, Wu et al. 2007). In this study, pirfenidone demonstrated an inhibitory effect on TGF-β1 mediated collagen accumulation in HFL1 cells in a dose-dependent manner. 10 and 100 μM was found not effective but 300 μM demonstrated statistical 22±7% inhibition (P<0.0001), 500 μM-33±8% inhibition (P<0.0001) and 1000 μM inhibited almost 100% (P<0.0001) (FIG. 1B)

NAC has been reported to modify TGF-β1 action by reducing an active 25 kDa dimer of TGF-β1 into inactive 12.5 kDa monomer thus abrogating TGF-β1 signaling (Lichtenberger, Montague et al. 2006). In this study, the inventors have documented that NAC is capable of inhibiting TGF-β1 mediated extracellular matrix accumulation in HFL1 cells, although millimolar (mM) concentrations are required to produce antifibrotic effect. 0.1, 0.5 and 1 mM were not effective whereas 2 mM of NAC caused 32±14% suppression (P<0.005), 5 mM-45±17% (P<0.005), 10 mM-70.5±18% (P<0.001), and 20 mM-100% inhibition (P<0.0001) (FIG. 1C).

Next, the inventors tested the effect of the combinational compositions of tranilast with NAC and pirfenidone with NAC. The inventors found that the combination of tranilast with NAC has substantial antifibrotic activity against TGF-β1 stimulated HFL1 cells. Also, the combination of pirfenidone with NAC was more effective. Thus, combination compositions of tranilast with NAC or pirfenidone with NAC could be very useful for the treatment of fibrotic disorders.

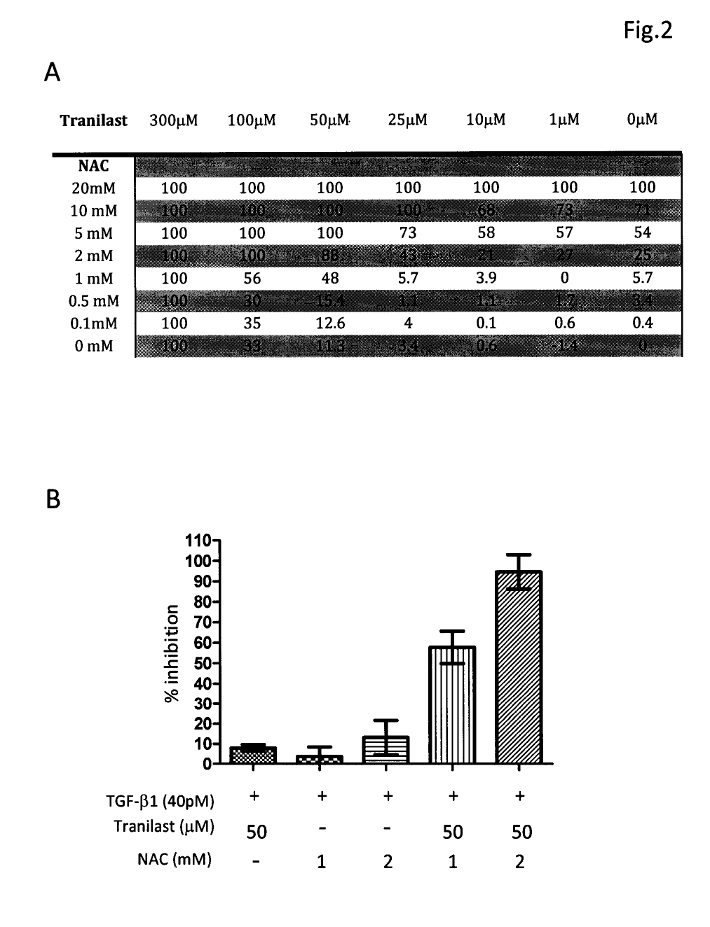

Together, tranilast and NAC were able to suppress ECM accumulation to a greater extent than either compound alone. Specifically, as seen in FIG. 2A, tranilast at maximal therapeutic dose (300 μM) can suppress TGF-β1 mediated collagen accumulation by 100%. The same level of suppression can be achieved by only 100 μM in combination with 2 mM NAC or by only 25 μM of tranilast by combining with 10 mM NAC. Further, the addition of 2 mM NAC to 50 μM of tranilast resulted in super-additive and synergistic effect (88%), compared to tranilast alone (11%) and NAC alone (25%). This represents a shift in the potency of tranilast of 8 fold. Strong synergistic enhancement of tranilast by NAC was confirmed in the second run (FIG. 2B) where the assay was performed with 6 replicates to confirm the observation.

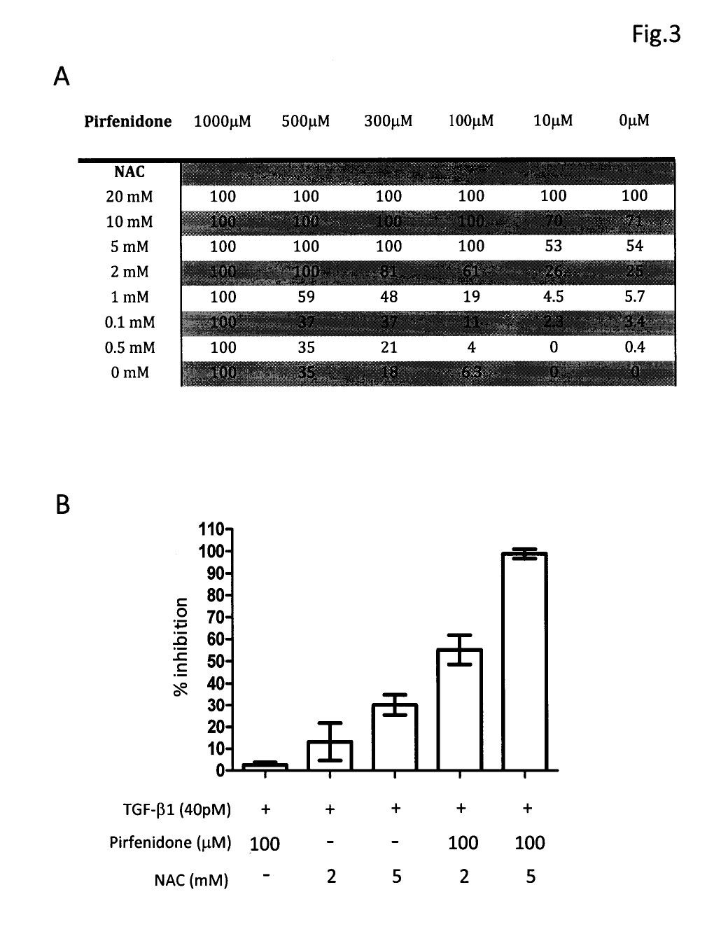

The combination of lower doses of pirfenidone and NAC was more effective. As seen in FIG. 3A, 100% anti-fibrosis activity can be achieved by using maximal therapeutic dose of pirfenidone, 1000 μM. However, the same level of suppression is achievable by only 100 μM of pirfenidone in combination with 5 mM of NAC. 300 μM of pirfenidone alone can only cause inhibition by 18%. By means of combination with 2 mM of NAC potency of pirfenidone has shifted to 81%. The combination of low doses of pirfenidone and NAC, therefore, results in the inhibition of TGF-β1 induced collagen accumulation to levels previously unattainable by either compound alone. FIG. 3B demonstrates results of the second run screen where 6 replicates were used in the assay.

As will be apparent to those skilled in the art in the light of the foregoing disclosure, many alterations and modifications are possible in the practice of the invention without departing from the spirit or scope thereof. It is therefore intended that the following appended claims and claims hereafter introduced are interpreted to include all such alterations and modifications as are within their true scope.

### REFERENCES

Azuma, A., T. Nukiwa, et al. (2005). “Double-blind, placebo-controlled trial of pirfenidone in patients with idiopathic pulmonary fibrosis.” Am J Respir Crit Care Med 171(9): 1040-7.

Demedts, M., J. Behr, et al. (2005). “High-dose acetylcysteine in idiopathic pulmonary fibrosis.” N Engl J Med 353(21): 2229-42.

Holmes, D., P. Fitzgerald, et al. (2000). “The PRESTO (Prevention of restenosis with tranilast and its outcomes) protocol: a double-blind, placebo-controlled trial.” Am Heart J 139(1 Pt 1): 23-31.

Kusama, H., S. Kikuchi, et al. (1999). “Tranilast inhibits the proliferation of human coronary smooth muscle cell through the activation of p2lwaf1.” Atherosclerosis 143(2): 307-13.

Lichtenberger, F. J., C. Montague, et al. (2006). “NAC and DTT promote TGF-betal monomer formation: demonstration of competitive binding.” J Inflamm (Lond) 3: 7.

Shi, S., J. Wu, et al. (2007). “Single- and multiple-dose pharmacokinetics of pirfenidone, an antifibrotic agent, in healthy Chinese volunteers.” J Clin Pharmacol 47(10): 1268-76.

Whyte, I. M., B. Francis, et al. (2007). “Safety and efficacy of intravenous N-acetylcysteine for acetaminophen overdose: analysis of the Hunter Area Toxicology Service (HATS) database.” Curr Med Res Opin 23(10): 2359-68.

We claim:

1. A method of treating, preventing or reducing a fibroproliferative disorder in a mammal comprising administering a combination of a pharmacologically effective dose of cytokine modifier and a pharmacologically effective dose of an anti-oxidant which is a precursor of glutathione.

2. A method according to claim 1 wherein the cytokine modifier is tranilast, or a pharmaceutically acceptable derivative, salt, metabolite, or structural or functional analogue thereof.

3. A method according to claim 1 wherein the cytokine modifier is pirfenidone, or a pharmaceutically acceptable derivative, salt, metabolite, or structural or functional analogue thereof.

4. A method according to claim 1 wherein the anti-oxidant is N-acetyl-L-cysteine, or a pharmaceutically acceptable derivative, salt, metabolite, or structural or functional analogue thereof.

5. A method according to claim 1 wherein the cytokine modifier and the anti-oxidant compound are administered separately.

6. A method according to claim 1 wherein the cytokine modifier and the anti-oxidant are administered concurrently.

7. A method according to claim 1 wherein the cytokine modifier is tranilast, or a pharmaceutically acceptable derivative, salt, metabolite, or structural or functional analogue thereof; and wherein the anti-oxidant is N-acetyl-L-cysteine, or a pharmaceutically acceptable derivative, salt, metabolite, or structural or functional analogue thereof.

8. A method according to claim 1 wherein the cytokine modifier is pirfenidone, or a pharmaceutically acceptable derivative, salt, metabolite, or structural or functional analogue thereof; and wherein the anti-oxidant is N-acetyl-L-cysteine, or a pharmaceutically acceptable derivative, salt, metabolite, or structural or functional analogue thereof

9. A method according to claim 7 wherein the daily dose of tranilast is in the range of 100 mg to 600 mg; and wherein the daily dose of N-acetyl-L-cysteine is in the range of 200 mg to 1800 mg.

10. A method according to claim 8 wherein the daily dose of pirfenidone is in the range of 300 mg to 1800 mg; and wherein the daily dose of N-acetyl-L-cysteine is in the range of 200 mg to 1800 mg.

11. A method according to claim 1 wherein both the cytokine modifier and the anti-oxidant are administered by any suitable means for oral, parenteral, rectal, cutaneous, nasal, vaginal, or inhalant use.

12. A method according to claim 1 wherein either or both of the cytokine modifier and the anti-oxidant are admixed with a pharmaceutical carrier before administration.

13. A composition comprising a pharmacologically effective dose of a cytokine modifier and a pharmacologically effective dose of an anti-oxidant which is a precursor of glutathione.

14. A composition comprising a pharmacologically effective dose of tranilast and a pharmacologically effective dose of N-acetyl-Lcysteine.

15. A composition comprising a pharmacologically effective dose of pirfenidone and a pharmacologically effective dose of N-acetyl-Lcysteine.

16. A composition according to claim 13 wherein the cytokine modifier and the anti-oxidant are in dosage unit form.

17. A composition according to claim 16 wherein the composition is in the form of a tablet, capsule, granule, powder, syrup, suspension, emulsion, solution, gel, paste, ointment, cream, lotion, plaster, skin patch, drench, suppository, enema, injectable, implant, spray or aerosol.

18. A composition according to claim 16 further comprising a pharmaceutically acceptable carrier.

19. A composition according to claim 14 wherein the pharmacologically effective dose of tranilast and the pharmacologically effective dose of N-acetyl-L-cysteine are effective in combination to treat a fibroproliferative disorder.

20. A composition according to claim 19 wherein the pharmacologically effective dose of tranilast is below a dose of tranilast that would be pharmacologically effective if the tranilast were administered in isolation, and wherein the pharmacologically effective dose of N-acetyl-L-cysteine is below a dose of N-acetyl-L-cysteine that would be pharmacologically effective if the N-acetyl-L-cysteine were administered in isolation.

21. A composition according to claim 15 wherein the pharmacologically effective dose of pirfenidone and the pharmacologically effective dose of N-acetyl-L-cysteine are effective in combination to treat a fibroproliferative disorder.

22. A composition according to claim 21 wherein the pharmacologically effective dose of pirfenidone is below a dose of pirfenidone that would be pharmacologically effective if the pirfenidone were administered in isolation, and wherein the pharmacologically effective dose of N-acetyl-L-cysteine is below a dose of N-acetyl-L-cysteine that would be pharmacologically effective if the N-acetyl-L-cysteine were administered in isolation.

23. A method according to claim 1 wherein the fibroproliferative disorder is one of pulmonary fibrosis, liver fibrosis, kidney fibrosis, uterine fibrosis, vascular fibrosis, or interventional therapy triggered fibrosis.

24. A method according to claim 1 wherein the pharmacologically effective dose of the cytokine modifier and the pharmacologically effective dose of the anti-oxidant are effective in combination to treat the fibroproliferative disorder.
